(19) United States
(12) Patent Application Publication
(10) Pub. No.: US 2007/0100183 A1
(43) Pub. Date: May 3, 2007

(54) Alkylation of aromatic compounds

(76) Inventors: Mark Andrew Harmer, Kennett Square, PA (US); Christopher P. Junk, Wilmington, DE (US); Jemma Vickery, Hants (GB); Zoe Schnepp, Beech Hill (GB)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/583,265

(22) Filed: Oct. 19, 2006

Related U.S. Application Data
(60) Provisional application No. 60/730,803, filed on Oct. 27, 2005.

(51) Int. Cl. C07C 13/28; C07C 2/02; C07C 6/12

(52) U.S. Cl. 585/422

(57) ABSTRACT

The present invention relates to the synthesis of alkylated aromatic compounds. Alkylated aromatic compounds are synthesized by reacting an aromatic compound with a monoolefin in the presence of a porous microcomposite comprising at least one fluorinated sulfonic acid on silica.

### FIELD OF INVENTION

This invention relates to a process for making alkylated aromatic compounds.

### BACKGROUND

The alkylation of aromatic compounds such as benzene and benzene derivatives with olefins is carried out on a large scale in the chemical industry (Perego and Ingallina (Catalysis Today (2002) 73:3-22) and Almeida, et al. (J. Am. Oil Chem. Soc. (1994) 71:675-694). Alkyl benzenes have many industrial uses. For example, ethyl benzene, formed by the reaction of ethylene with benzene, is an intermediate in styrene production. Alkylation of benzene with propylene yields cumene, an intermediate in phenol and acetone production. Linear alkyl benzenes are synthesized from the reaction of longer-chain olefins (ca. 10-18 carbon atoms) with benzene or benzene derivatives; the linear alkyl benzenes are then sulfonated to produce surfactants.

Historically, aromatic alkylation reactions have been carried out in the presence of a homogeneous (i.e., soluble) acid catalyst. Homogeneous catalysts, while effective, produce highly corrosive media with chemically reactive waste streams. Thus, there has been considerable effort to replace homogeneous catalysts with cost-effective and active solid acid catalysts, which allow for simpler product purification and safer process operation.

A. de Angelis, et al. (Catalysis Today, 2001, 65:353-371) describe the use of a solid acid catalyst prepared by treating amorphous silica gel with trifluoromethanesulfonic acid to catalyze the alkylation of isobutane with n-butenes.

### SUMMARY OF THE INVENTION

The present invention provides a method for carrying out aromatic alkylation reactions using a porous solid catalyst comprised of at least one fluorinated sulfonic acid on silica.

The present invention relates to a process for making at least one alkylated aromatic compound of the Formula: 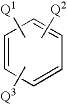
wherein:

a) Q¹ is H, —CH₃, —C₂H₅, or CH₃—CH—CH₃;

b) Q² is H, —CH₃ or —C₂H₅; and

c) Q³ is —C₂H₅ or C₃ to C₁₈ straight chain alkyl group having therein a single CH group, the carbon atom of which is bonded to the aromatic compound;
by a process comprising reacting a C₂ to C₁₈ straight-chain monoolefin with an aromatic compound of the Formula: 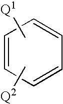
wherein Q₁ and Q² are as defined above;
in the presence of at least one porous microcomposite comprising at least one fluorinated sulfonic acid and silica made by a process comprising the steps of:
(a) contacting, in the presence of water:
   (1) at least one silica precursor;
   (2) at least one fluorosulfonic acid selected from the group consisting of:
      (i) 1,1,2,2-tetrafluoroethanesulfonic acid;
      (ii) 1,1,2-trifluoro-2-(perfluoroethoxy)ethanesulfonic acid;
      (iii) 1,1,2-trifluoro-2-(trifluoromethoxy)ethanesulfonic acid
      (iv) 1,1,2-trifluoro-2-(perfluoropropoxy)ethanesulfonic acid;
      (v) 1,1,2,3,3,3-hexafluoropropanesulfonic acid; and
      (vi) 2-chloro-1,1,2-trifluoroethanesulfonic acid; and optionally at least one inorganic acid; and
   (3) optionally, a non-reacting solvent; to form a mixture;
(b) aging the mixture to form a gelled mixture; and
(c) drying the gelled mixture to remove substantially all water and non-reacting solvent, if any, therein;
said reacting being carried out at a temperature between about 25° C. and about 200° C., and a pressure between atmospheric pressure and that pressure required to maintain the reactants in a liquid state.

The present invention also relates to a process for making at least one alkylated aromatic compound of the Formula: 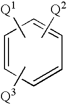
wherein:

a) Q¹ is H, —CH₃, —C₂H₅, or CH₃—CH—CH₃;

b) Q² is H, —CH₃ or—C₂H₅; and

c) Q³ is —C₂H₅ or C₃ to C₁₈ straight chain alkyl group having therein a single CH group, the carbon atom of which is bonded to the aromatic compound;
by a process comprising reacting a C₂ to C₁₈ straight-chain monoolefin with an aromatic compound of the Formula: 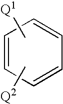
wherein Q¹ and Q² are as defined above;
in the presence of at least one porous microcomposite comprising at least one fluorinated sulfonic acid and silica made by a process comprising the steps of:
(a) contacting at least one fluorosulfonic acid selected from the group consisting of:
   (i) 1,1,2,2-tetrafluoroethanesulfonic acid;
   (ii) 1,1,2-trifluoro-2-(perfluoroethoxy)ethanesulfonic acid;
   (iii) 1,1,2-trifluoro-2-(trifluoromethoxy)ethanesulfonic acid;
   (iv) 1,1,2-trifluoro-2-(perfluoropropoxy)ethanesulfonic acid;
   (v) 1,1,2,3,3,3-hexafluoropropanesulfonic acid; and
   (vi) 2-chloro-1,1,2-trifluoroethanesulfonic acid;
optionally in a non-reacting solvent, with a preformed porous silica support;
(b) drying the acid-impregnated porous silica to remove therefrom substantially all of the non-reacting solvent and water, if any, contained therein;
said reacting being carried out at a temperature between about 25° C. and about 200° C., and a pressure between atmospheric pressure and that pressure required to maintain the reactants in a liquid state.

### BRIEF DESCRIPTION OF THE FIGURES

FIG. 1 is a GC tracing of the products obtained from the alkylation of p-xylene with 1-dodecene using the microcomposite HCF₂CF₂SO₃H on silica.

FIG. 2 is a GC tracing of the products obtained from the alkylation of p-xylene with 1-dodecene using HCF₂CF₂SO₃H (without silica).

### DETAILED DESCRIPTION OF THE INVENTION

The present invention relates to a process for alkylating aromatic compounds with monoolefins using as the catalyst a porous microcomposite comprising at least one fluorinated sulfonic acid on silica.

Definitions

In this disclosure, a number of terms and abbreviations are used. The following definitions are provided.

By “alkyl” is meant a monovalent radical having the general Formula CₙH₂ₙ₊₁. “Monovalent” means having a valence of one.

By “hydrocarbyl” is meant a monovalent group containing only carbon and hydrogen.

By “catalyst” is meant a substance that affects the rate of the reaction but not the reaction equilibrium, and emerges from the process chemically unchanged.

The present invention relates to a process for making at least one alkylated aromatic compound of the Formula: 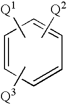
wherein:

a) Q₁ is H, —CH₃, —C₂H₅, or CH₃—CH—CH₃;

b) Q² is H, —CH₃ or —C₂H₅; and

c) Q³ is —C₂H₅ or C₃ to C₁₈ straight chain alkyl group having therein a single CH group, the carbon atom of which is bonded to the aromatic compound;
by a process comprising reacting a C₂ to C₁₈ straight-chain monoolefin with an aromatic compound of the Formula: 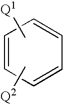
wherein Q₁ and Q² are as defined above;
in the presence of at least one porous microcomposite comprising at least one fluorinated sulfonic acid and silica made by a process comprising the steps of:
(a) contacting, in the presence of water:
   (1) at least one silica precursor;
   (2) at least one fluorosulfonic acid selected from the group consisting of:
      (i) 1,1,2,2-tetrafluoroethanesulfonic acid;
      (ii) 1,1,2-trifluoro-2-(perfluoroethoxy)ethanesulfonic acid;
      (iii) 1,1,2-trifluoro-2-(trifluoromethoxy)ethanesulfonic acid;
      (iv) 1,1,2-trifluoro-2-(perfluoropropoxy)ethanesulfonic acid;
      (v) 1,1,2,3,3,3-hexafluoropropanesulfonic acid; and
      (vi) 2-chloro-1,1,2-trifluoroethanesulfonic acid; and optionally at least one inorganic acid; and
   (3) optionally, a non-reacting solvent; to form a mixture;
(b) aging the mixture to form a gelled mixture; and
(c) drying the gelled mixture to remove substantially all water and non-reacting solvent, if any, therein.

In one embodiment of the invention, Q₁ and Q² are both H.

Preparation of the Porous Microcomposite

The term “silica precursor” refers to a silicon and oxygen-containing compound capable of forming silica in the presence of water. For example, it is well known that a range of silicon alkoxides of the Formula Si(OR)₄, wherein R is —CH₃, —C₂H₅, or C3 to C6 straight-chain or branched alkyl, can be hydrolyzed and condensed to form a silica network. A silica network is a known concept in the art and is described in Brinker, C. J. and G. W. Scherer, Sol-Gel Science (Academic Press, NY, 1990). Preferably R is methyl or ethyl. Such precursors include tetramethoxysilane (tetramethyl orthosilicate), tetraethoxysilane (tetraethyl orthosilicate), tetrapropoxysilane, tetrabutoxysilane. Also included as a silica precursor is silicon tetrachloride. Further silica precursors comprise organically modified silica, for example, CH₃Si(OCH₃)₃, PhSi(OCH₃)₃ where Ph is phenyl, and (CH₃)₂Si(OCH₃)₂. Other silica precursors include metal silicates, such as potassium silicate, sodium silicate, and lithium silicate. Potassium, sodium, or lithium ions can be removed using a cation exchange resin, such as DOWEX® (Dow Chemical, Midland, Mich.), that generates polysilicic acid which gels upon aging and drying.

An inorganic acid or a fluorinated sulfonic acid selected from the group consisting of 1,1,2,2-tetrafluoroethanesulfonic acid, 1,1,2-trifluoro-2-(perfluoroethoxy)ethanesulfonic acid, 1,1,2-trifluoro-2-(trifluoromethoxy)ethanesulfonic acid, 1,1,2-trifluoro-2-(perfluoropropoxy)ethanesulfonic acid, 1,1,2,3,3,3-hexafluoropropanesulfonic acid, and 2-chloro-1,1,2-trifluoroethanesulfonic acid may be used to hydrolyze silicon alkoxides or organically modified silicon alkoxides. Suitable inorganic acids include hydrochloric acid, sulfuric acid, and nitric acid.

The at least one fluorinated sulfonic acid may be synthesized as described in the following references: U.S. Pat. No. 2,403,207, Rice, et al. (Inorg. Chem., 1991, 30:4635-4638), Coffman, etal. (J. Org. Chem., 1949, 14:747-753 and Koshar, etal. (J. Am. Chem. Soc. (1953) 75:4595-4596), and can be used in either hydrated or anhydrous forms.

The non-reacting solvent may be a lower aliphatic alcohol such as methanol, 1-propanol, 2-propanol, and n-butanol. Other suitable solvents include acetonitrile, diethyl ether, dimethyl formamide, dimethylsulfoxide, nitromethane, tetrahydrofuran and acetone.

Aging of the mixture may be carried out under air. Alternatively, the mixture may be aged under a flowing, non-reactive gas such as argon, nitrogen or helium, or under a vacuum. The temperature for aging of the mixture may be from about 15° C. to about 150° C. Gelation of the mixture will be dependent on a number of factors such as the amount of water present, temperature, solvent, concentrations, and the acid or acids used. See Brinker, C. J. and G. W. Scherer, supra, pages 518-523 for a discussion of silica gel formation.

Drying of the gelled mixture to remove substantially all remaining water and/or alcohol can be carried out as described for aging. The gelled mixture is preferably dried under an inert gas such as nitrogen at a temperature from about 50° C. to about 150° C.

The microcomposite of the present invention exists as a particulate solid that is glass-like in nature, typically 0.1 to 4 millimeters in size and structurally hard, similar to dried silica gels. The porous nature of the material is evident from the high surface areas measured for these glass-like pieces. Typical pore diameters are in the range of about 0.5 to about 75 nanometers; preferably the pore diameters are in the range of about 0.5 to about 25 nanometers. The weight percentage of fluorinated sulfonic acid relative to silica is from about 0.1% to about 90%. Optionally, the hard glass-like product can be comminuted, such as by grinding with a pestle and mortar.

In another embodiment, the porous microcomposite used in the alkylation reaction is prepared from a preformed silica support. Thus, the present invention also provides a process for making at least one alkylated aromatic compound of the Formula: 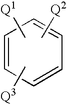
wherein:

a) Q₁ is H, —CH₃, —C₂H₅, or CH₃—CH—CH₃;

b) Q² is H, —CH₃ or —C₂H₅; and

c) Q³ is —C₂H₅ or C₃ to C₁₈ straight chain alkyl group having therein a single CH group, the carbon atom of which is bonded to the aromatic compound;
by a process comprising reacting a C₂ to C₁₈ straight-chain monoolefin with an aromatic compound of the Formula: 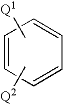
wherein Q₁ and Q² are as defined above;
in the presence of at least one porous microcomposite comprising at least one fluorinated sulfonic acid and silica made by a process comprising the steps of:
(a) contacting at least one fluorosulfonic acid selected from the group consisting of:
   (i) 1,1,2,2-tetrafluoroethanesulfonic acid;
   (ii) 1,1,2-trifluoro-2-(perfluoroethoxy)ethanesulfonic acid;
   (iii) 1,1,2-trifluoro-2-(trifluoromethoxy)ethanesulfonic acid;
   (iv) ,1 ,2-trifluoro-2-(perfluoropropoxy)ethanesulfonic acid
   (v) 1,1,2,3,3,3-hexafluoropropanesulfonic acid; and
   (vi) 2-chloro-1,1,2-trifluoroethanesulfonic acid;
   optionally in a non-reacting solvent, with a preformed porous silica support;
(b) allowing sufficient time for at least some of the at least one fluorosulfonic acid to be absorbed by the support to form an acid-impregnated silica;
(c) drying the acid-impregnated porous silica to remove therefrom substantially all of the non-reacting solvent and water, if any, contained therein;
at a temperature between about 25° C. and about 200° C., and a pressure between atmospheric pressure and that pressure required to maintain the reactants in a liquid state, wherein at the start of the reaction the aromatic compound is in molar excess relative to the monoolefin.

The preformed porous silica support may be obtained commercially from, for example, PQ Corporation (Valley Forge, Pa.), W. R. Grace (Baltimore, Md.) or Aldrich (St. Louis, Mo.). An example is Silica Gel Beads (2-3 millimeter amorphous silicon dioxide beads) from PQ Corporation.

The non-reacting solvent may be a lower aliphatic alcohol such as methanol, 1-propanol, 2-propanol, and n-butanol. Other suitable solvents include acetonitrile, diethyl ether, dimethyl formamide, dimethylsulfoxide, nitromethane, tetrahydrofuran and acetone.

Drying of the acid-impregnated porous silica may be carried out under air. Alternatively, the acid-impregnated porous silica may be aged under a flowing, non-reactive gas such as argon, nitrogen or helium, or under a vacuum. The temperature for drying is from about 15° C. to about 150° C. Preferably the acid-impregnated porous silica is dried under an inert gas such as nitrogen at a temperature from about 50° C. to about 150° C.

The weight percentage of fluorinated sulfonic acid relative to silica is from about 0.1% to about 90%; the weight percent of the fluorinated sulfonic acid will depend on the pore volume of the preformed support.

It is believed that the highly porous structure of the microcomposite comprises a continuous silicon oxide phase that absorbs the highly dispersed fluorinated sulfonic acid catalyst within and throughout a connected network of porous channels. The porous nature of the material can be readily demonstrated, for example, by solvent absorption. The microcomposite can be observed to emit bubbles, which are evolved due to the displacement of the air from within the porous network.

The porous microcomposite is used in the aromatic alkylation reaction at a concentration of from about 0.01% to about 20% by weight of the reaction solution comprising the aromatic compound and the monoolefin. In a more specific embodiment, the porous microcomposite is used at a concentration of from about from about 0.1% to about 10%. In an even more specific embodiment, the porous microcomposite is used at a concentration of from about 0.1% to about 5%.

The aromatic compound used in the alkylation reaction is benzene or a benzene-derivative, such as toluene, xylene, ethyl benzene or isopropyl benzene.

The alkylation reaction is carried out at a temperature between about 25° C. and about 200° C., and a pressure between atmospheric pressure and that pressure required to maintain the reactants in a liquid state. In one embodiment of the invention, the reaction is carried out at about 25° C. and the pressure is atmospheric pressure.

The molar ratio of aromatic compound to monoolefin will depend upon the desired reaction product, i.e. whether monoadduct or the addition of two or more alkyl groups to the aromatic compound is the object of the reaction. If monoadduct is the desired product, a molar excess of the aromatic preferably is used, more preferably at least about 3:1 aromatic compound to monoolefin, still more preferably at least about 4:1, and most preferably at least about 8:1.

The aromatic alkylation reaction may be carried out in batch, sequential batch (i.e., a series of batch reactors) or in continuous mode in any of the equipment customarily employed for continuous process (see for example, H. S. Fogler, Elementary Chemical Reaction Engineering, Prentice-Hall, Inc., N.J., USA). One skilled in the art will recognize that at higher temperatures or pressures a sealed vessel or pressure vessel is required.

The alkylated aromatic product(s) may be recovered from the porous microcomposite by any suitable method known to those skilled in the art, including decantation. The porous microcomposite may be reused in subsequent reactions.

### EXAMPLES

### General Materials and Methods

The following abbreviations are used:

Nuclear magnetic resonance is abbreviated NMR; gas chromatography is abbreviated GC; gas chromatography-mass spectrometry is abbreviated GC-MS; thin layer chromatography is abbreviated TLC; thermogravimetric analysis (using a Universal V3.9A TA instrument analyzer (TA Instruments, Inc., Newcastle, Del.)) is abbreviated TGA. Centigrade is abbreviated C, megapascal is abbreviated MPa, gram is abbreviated g, kilogram is abbreviated Kg, milliliter(s) is abbreviated ml, hour is abbreviated hr; weight percent is abbreviated wt %; milliequivalents is abbreviated meq; melting point is abbreviated Mp; differential scanning calorimetry is abbreviated DSC.

Acetonitrile, oleum (20% SO₃), sodium sulfite (Na₂SO₃, 98%), and acetone were obtained from Acros (Hampton, N.H.). Potassium metabisulfite (K₂S₂O₅, 99%), was obtained from Mallinckrodt Laboratory Chemicals (Phillipsburg, N.J.). Tetramethyl orthosilicate, tetraethyl orthosilicate HCl, p-xylene, potassium sulfite hydrate (KHSO₃.H₂O, 95%), sodium bisulfite (NaHSO₃), diethyl ether, trifluoromethanesulfonic acid, and 1-dodecene were obtained from Aldrich (St. Louis, Mo.). Sulfuric acid was obtained from EMD Chemicals, Inc. (Gibbstown, N.J.). Perfluoro(ethyl vinyl ether), perfluoro(methyl vinyl ether), hexafluoropropene and tetrafluoroethylene were obtained from DuPont Fluoroproducts (Wilmington, Del.). 1,1,2,2-Tetrafluoro-2-(pentafluoroethoxy)sulfonate was obtained from SynQuest Laboratories, Inc. (Alachua, Fla.).

Preparation of Fluorosulfonic Acid Precursors

(A) Synthesis of potassium 1,1,2.2-tetrafluoroethanesulfonate (TFES-K):

A 1-gallon Hastelloy® C276 reaction vessel was charged with a solution of potassium sulfite hydrate (176 g, 1.0 mol), potassium metabisulfite (610 g, 2.8 mol) and deionized water (2000 ml). The pH of this solution was 5.8. The vessel was cooled to 18° C., evacuated to 0.10 MPa, and purged with nitrogen. The evacuate/purge cycle was repeated two more times. To the vessel was then added tetrafluoroethylene (TFE, 66 g), and it was heated to 100° C. at which time the inside pressure was 1.14 MPa. The reaction temperature was increased to 125° C. and kept there for 3 hr. As the TFE pressure decreased due to the reaction, more TFE was added in small aliquots (20-30 g each) to maintain operating pressure roughly between 1.14 and 1.48 MPa. Once 500 g (5.0 mol) of TFE had been fed after the initial 66 g precharge, the vessel was vented and cooled to 25° C. The pH of the clear light yellow reaction solution was 10-11. This solution was buffered to pH 7 through the addition of potassium metabisulfite (16 g).

The water was removed in vacuo on a rotary evaporator to produce a wet solid. The solid was then placed in a freeze dryer (Virtis Freezemobile 35xl; Gardiner, N.Y.) for 72 hr to reduce the water content to approximately 1.5 wt % (1387 g crude material). The theoretical mass of total solids was 1351 g. The mass balance was very close to ideal and the isolated solid had slightly higher mass due to moisture. This added freeze drying step had the advantage of producing a free-flowing white powder whereas treatment in a vacuum oven resulted in a soapy solid cake that was very difficult to remove and had to be chipped and broken out of the flask.

The crude TFES-K can be further purified and isolated by extraction with reagent grade acetone, filtration, and drying.

¹⁹F NMR (D₂O).δ−122.0.(dt, J_{FH}=6 Hz, J_{FF}=6 Hz, 2F); -136.1 (dt, J_{FH}=53 Hz, 2F). ¹H NMR (D₂O) δ66.4 (tt, J_{FH}=53 Hz, J_{FH}=6 Hz, 1 H). % Water by Karl-Fisher titration: 580 ppm. Analytical calculation for C₂HO₃F₄SK: C, 10.9: H, 0.5: N, 0.0. Experimental results: C, 11.1: H, 0.7: N, 0.2. Mp (DSC): 242° C. TGA (air): 10% wt. loss @ 367° C., 50% wt. loss @ 375° C. TGA (N₂): 10% wt. loss @ 363° C., 50% wt. loss @ 375° C.

(B) Synthesis of potassium-11,2-trifluoro-2-(perfluoroethoxv)ethanesulfonate (TPES-K):

A 1-gallon Hastelloye C276 reaction vessel was charged with a solution of potassium sulfite hydrate (88 g, 0.56 mol), potassium metabisulfite (340 g, 1.53 mol) and deionized water (2000 ml). The vessel was cooled to 7° C., evacuated to 0.05 MPa, and purged with nitrogen. The evacuate/purge cycle was repeated two more times. To the vessel was then added perfluoro(ethyl vinyl ether) (PEVE, 600 g, 2.78 mol), and it was heated to 125° C. at which time the inside pressure was 2.31 MPa. The reaction temperature was maintained at 125° C. for 10 hr. The pressure dropped to 0.26 MPa at which point the vessel was vented and cooled to 25° C. The crude reaction product was a white crystalline precipitate with a colorless aqueous layer (pH =7) above it.

The ¹⁹F NMR spectrum of the white solid showed pure desired product, while the spectrum of the aqueous layer showed a small but detectable amount of a fluorinated impurity. The desired product is less soluble in water so it precipitated in pure form.

The product slurry was suction filtered through a fritted glass funnel, and the wet cake was dried in a vacuum oven (60° C., 0.01 MPa) for 48 hr. The product was obtained as off-white crystals (904 g, 97% yield).

¹⁹F NMR (D₂O) δ −86.5.(s, 3F); −89.2, −91.3 (subsplit ABq, J_{FF}=147 Hz, 2F); −119.3, −121.2 (subsplit ABq, J_{FF}=258 Hz, 2F); −144.3 (dm, J_{FH}=53 Hz, 1F). ¹H NMR (D₂O) δ 6.7 (dm, J_{FH}=53 Hz, 1H). Mp (DSC) 263° C. Analytical calculation for C₄HO₄F₈SK: C, 14.3: H, 0.3 Experimental results: C, 14.1: H, 0.3. TGA (air): 10% wt. loss @ 359° C., 50% wt. loss @ 367° C. TGA (N₂): 10% wt. loss @ 362° C., 50% wt. loss @ 374° C.

(C) Synthesis of potassium-1,1,2-trifluoro-2-(trifluoromethoxv)ethanesulfonate (TTES-K)

A 1-gallon Hastelloy® C276 reaction vessel was charged with a solution of potassium sulfite hydrate (114 g, 0.72 mol), potassium metabisulfite (440 g, 1.98 mol) and deionized water (2000 ml). The pH of this solution was 5.8. The vessel was cooled to −35° C., evacuated to 0.08 MPa, and purged with nitrogen. The evacuate/purge cycle was repeated two more times. To the vessel was then added perfluoro(methyl vinyl ether) (PMVE, 600 g, 3.61 mol) and it was heated to 125° C. at which time the inside pressure was 3.29 MPa. The reaction temperature was maintained at 125° C. for 6 hr. The pressure dropped to 0.27 MPa at which point the vessel was vented and cooled to 25° C. Once cooled, a white crystalline precipitate of the desired product formed leaving a colorless clear aqueous solution above it (pH=7).

The ¹⁹F NMR spectrum of the white solid showed pure desired product, while the spectrum of the aqueous layer showed a small but detectable amount of a fluorinated impurity.

The solution was suction filtered through a fritted glass funnel for 6 hr to remove most of the water. The wet cake was then dried in a vacuum oven at 0.01 MPa and 50° C. for 48 hr. This gave 854 g (83% yield) of a white powder. The final product was pure (by ¹⁹F and ¹H NMR) since the undesired byproduct remained in the water during filtration.

¹⁹F NMR (D₂O) δ −59.9.(d, J_{FH}=4 Hz, 3F); −119.6, −120.2 (subsplit ABq, J=260 Hz, 2F); −144.9 (dm, J_{FH}=53 Hz, 1 F). ¹H NMR (D₂O) δ 6.6 (dm, J_{FH}=53 Hz, 1 H). Water by Karl-Fisher titration: 71 ppm. Analytical calculation for C₃HF₆SO₄K: C, 12.6: H, 0.4: N, 0.0 Experimental results: C, 12.6: H, 0.0: N, 0.1. Mp (DSC) 257° C. TGA (air): 10% wt. loss @ 343° C., 50% wt. loss @ 358° C. TGA (N₂): 10% wt. loss @ 341° C., 50% wt. loss @ 357° C.

(D) Synthesis of sodium 1,1,2,3.3,3-hexafluoropropanesulfonate (HFPS-Na)

A 1-gallon Hastelloy® C reaction vessel was charged with a solution of anhydrous sodium sulfite (25 g, 0.20 mol), sodium bisulfite 73 g, (0.70 mol) and of deionized water (400 ml). The pH of this solution was 5.7. The vessel was cooled to 4° C., evacuated to 0.08 MPa, and then charged with hexafluoropropene (HFP, 120 g, 0.8 mol, 0.43 MPa). The vessel was heated with agitation to 120° C. and kept there for 3 hr. The pressure rose to a maximum of 1.83 MPa and then dropped down to 0.27 MPa within 30 minutes. At the end, the vessel was cooled and the remaining HFP was vented, and the reactor was purged with nitrogen. The final solution had a pH of 7.3.

The water was removed in vacuo on a rotary evaporator to produce a wet solid. The solid was then placed in a vacuum oven (0.02 MPa, 140° C., 48 hr) to produce 219 g of white solid which contained approximately 1 wt % water. The theoretical mass of total solids was 217 g.

The crude HFPS-Na can be further purified and isolated by extraction with reagent grade acetone, filtration, and drying.

¹⁹F NMR (D₂O) δ −74.5 (m, 3F); −113.1, −120.4 (ABq, J=264 Hz, 2F); −211.6 (dm, 1F). ¹H NMR (D₂O) δ 5.8 (dm, J_{FH}=43 Hz, 1 H). Mp (DSC) 126° C. TGA (air): 10% wt. loss @ 326° C., 50% wt. loss @ 446° C. TGA (N₂): 10% wt. loss @ 322° C., 50% wt. loss @ 449° C.

Preparation of Fluorosulfonic Acids

(E) Synthesis of 1,1.2.2-tetrafluoroethanesulfonic acid (TFESA)

A 100 ml round bottomed flask with a sidearm and equipped with a digital thermometer and magnetic stirr bar was placed in an ice bath under positive nitrogen pressure. To the flask was added 50 g crude TFES-K (from synthesis (A) above), 30 g of concentrated sulfuric acid (95-98%) and 78 g oleum (20 wt % SO₃) while stirring. The amount of oleum was chosen such that there would be a slight excess of SO₃ after the SO₃ reacted with and removed the water in the sulfuric acid and the crude TFES-K. The mixing caused a small exotherm, which was controlled by the ice bath. Once the exotherm was over, a distillation head with a water condenser was placed on the flask, and the flask was heated under nitrogen behind a safety shield. The pressure was slowly reduced using a PTFE membrane vacuum pump (Buchi V-500,) in steps of 100 Torr (13 kPa) in order to avoid foaming. A dry-ice trap was placed between the distillation apparatus and the pump to collect any excess SO₃. When the pot temperature reached 120° C. and the pressure was held at 20-30 Torr (2.7-4.0 kPa) a colorless liquid started to reflux which distilled at 110° C. and 31 Torr (4.1 kPa). A forerun of lower-boiling impurity (2.0 g) was obtained before collecting 28 g of the desired colorless acid, TFESA.

It was calculated that approximately 39.8 g TFES-K was present in the 50 g of impure TFES-K. Thus, the 28 g of product is an 85% yield of TFESA from TFES-K, as well as an 85% overall yield from TFE. Analysis gave the following results: ¹⁹F NMR (CD₃OD) −125.2dt, 3J_{FH}=6 Hz, 3J_{FF}=8Hz, 2F); −137.6 (dt, 2J_{FH}=53 Hz, 2F). ¹H NMR (CD3OD). 6.3 (tt, ³JFH =6 Hz, 2J_{FH}=53 Hz, 1H).

(F) Synthesis of 1,1,2,3,3,3-hexafluoropropanesulfonic acid (HFPSA)

A 100 ml round bottomed flask with a sidearm and equipped with a digital thermometer and magnetic stirr bar was placed in an ice bath under positive nitrogen pressure. To the flask was added 50 g crude sodium hexafluoropropanesulfonate (HFPS-Na) (from synthesis (D) above), 30 g of concentrated sulfuric acid (95-98%) and 58.5 g oleum (20 wt % SO₃) while stirring.

The amount of oleum was chosen such that there would be a slight excess of SO₃ after the SO₃ reacted with and removed the water in the sulfuric acid and the crude HFPSA. The mixing caused a small exotherm, which was controlled by the ice bath. Once the exotherm was over, a distillation head with a water condenser was placed on the flask, and the flask was heated under nitrogen behind a safety shield. The pressure was slowly reduced using a PTFE membrane vacuum pump in steps of 100 Torr (13 kPa) in order to avoid foaming. A dry-ice trap was placed between the distillation apparatus and the pump to collect any excess SO₃. When the pot temperature reached 100° C. and the pressure was held at 20-30 Torr (2.7-4 kPa) a colorless liquid started to reflux and later distilled at 118° C. and 23 Torr (3.1 kPa). A forerun of lower-boiling impurity (1.5 g) was obtained before collecting 36.0 g of the desired acid, hexafluoropropanesulfonic acid (HFPSA).

It was calculated that approximately 44 g HFPS-Na was present in 50 g of impure HFPS-Na. Thus, the 36.0 g of HFPSA product was an 89% yield from HFPS-Na, as well as an 84% overall yield from HFP.

¹9F NMR (D₂O) −74.5m, 3F); −113.1, −120.4 (ABq, J=264 Hz, 2F); −211.6 (dm, 1F). ¹H NMR (D₂O) 5.8 (dm, 2J_{FH}=43 Hz,1H).

(G) Synthesis of 2-chloro-1,1,2-trifluoroethanesulfonic acid

A 1-gallon Hastelloy® C276 reaction vessel was charged with a solution of 240 g sodium bisulfite hydrate (NaHSO₃.H2O, 95%), 128 g sodium metabisulfite (Na₂S₂O₅, 99%) and 800 ml of deionized water. The vessel was cooled to 18° C., evacuated to 0 kPa, and purged with nitrogen. The evacuate/purge cycle was repeated two more times. To the vessel was then added 233 g of chlorotrifluoroethylene in 50 g amounts until the last 33 g at a temperature of 125° C. which time the inside pressure is 250 psi. The reaction temperature was maintained at 125° C. for 3 hr, and then cooled to room temperature. The water was removed in vacuo on a rotary evaporator to produce a yellow/white solid which contained in part the sodium salt, CCIHFCF₂SO₃H. To 160 g of the yellow/white solid was added 250 ml of 98% sulfuric acid in a round bottomed flask. The mixture was heated and the acid monohydrate was distilled under vacuum at 119-120° C. (0.8 mm Hg). Thionyl chloride (70 ml) was then added to the acid monohydrate under a nitrogen atmosphere; the mixture was heated at 50° C. for one hour, and the excess thionyl chloride was removed under vacuum. The acid was removed by distillation under vacuum to give pure HCICFCF₂SO₃H, as shown by NMR.

Synthesis of Microcomposites Useful for the Invention:

(H) Preparation of Microcomposite of TFESA and Silica

Tetramethyl orthosilicate (4 g), water (4.7 g), and 0.04 M HCl (0.05 g) were stirred together for 15 minutes to hydrolyze the tetraalkoxide. HCF₂CF₂SO₃H (0.5 g) was then added, and the mixture was stirred for several hours. The resulting gel was left to dry in air in an uncovered beaker at room temperature for four days. Drying of the composite was completed in a 100° C. vacuum oven for 48 hours. The surface area, pore volume and pore diameter were determined by the Brunauer-Emmett-Teller (BET; see C. N. Satterfield, Heterogeneous Catalysis in Industrial Practice, 2^{nd} Edition, 1991, McGraw-Hill, Inc., NY, pages 134-139) method to be 565 m²/g, 0.32 cc/g and 2.3 nm.

(I) Preparation of Microcomposite of TFESA and Silica—Slow Drying

Tetramethyl orthosilicate (16 g), water (18.8 g) and 0.04 M HCl (0.2 g) were stirred together for 15 minutes to hydrolyze the tetraalkoxide. HCF₂CF₂SO₃H (2 g) was then added, and the mixture was stirred in a loosely capped jar for 72 hours to gel. The resulting gel was dried slowly in a 75° C. nitrogen oven (still in a loosely capped jar) for 7 days. Drying of the composite was completed in a 100° C. vacuum oven for 48 hours. The surface area, pore volume and pore diameter were determined to be 584 m²/g, 0.39 cc/g and 2.7 nm, respectively.

(J) Preparation of Microcomposite of TFESA and Silica—Rapid Drying

Tetramethyl orthosilicate (8 g), water (9.4 g) and 0.04 M HCl (0.1 g) were stirred together for 15 minutes to hydrolyze the tetraalkoxide. HCF₂CF₂SO₃H (1 g) was then added; the mixture was stirred for 1 minute to mix and then placed immediately in a 90° C. oven, in an open beaker under a nitrogen stream for 48 hours. Drying of the composite was completed in a 100° C. vacuum oven for 72 hours. The surface area, pore volume and pore diameter were determined by BET to be 506 m²/g, 0.29 cc/g and 2.3 nm respectively.

(K) Preparation of Microcomposite of TFESA and Silica

Tetramethyl orthosilicate (4 g), water (4.7 g) and 0.04 M HCl (0.05 g) were stirred together for 15 minutes to hydrolyze the tetraalkoxide. HCF₂CF₂SO₃H (1.59 g) was then added, and the mixture was stirred to gel (less than about two hours). The resulting gel was left to dry in air in an uncovered beaker at room temperature for four days. Drying of the composite was completed in a 100° C. vacuum oven for 48 hours. The composite comprised approximately 50% by weight of the acid relative to the weight of the silica. The surface area, pore volume and pore diameter were determined by BET to be 597 m²/g, 0.42 cc/g and 2.8 nm, respectively.

(L) Preparation of Microcomposite of TFESA and Silica

Tetramethyl orthosilicate (8 g), water (9.4 g) and 0.04 M HCl (0.1 g) were stirred together for 15 minutes to hydrolyze the tetraalkoxide. HCF₂CF₂SO₃H (0.45 g) was then added, and the mixture was stirred to gel (less than about two hours). The resulting gel was left to dry in air in an uncovered beaker at room temperature for four days. Drying of the composite was completed in a 100° C. vacuum oven for 48 hours. The composite comprised approximately 12.5% by weight of the acid relative to the weight of the silica. The surface area, pore volume and pore diameter were determined by BET to be 576 m²/g, 0.25 cc/g and 1.4 nm, respectively.

(M) Preparation of Microcomposite of TFESA and Silica

Tetramethyl orthosilicate (16 g), water (18.8 g) and 0.04 M HCl (0.2 g) were stirred together for 15 minutes to hydrolyze the tetraalkoxide. HCF₂CF₂SO₃H (0.33 g) was then added, and the mixture was stirred to gel (less than about two hours). The resulting gel was left to dry in air in an uncovered beaker at room temperature for four days. Drying of the composite was completed in a 100° C. vacuum oven for 48 hours. The composite comprised approximately 5% by weight of the acid relative to the weight of the silica. The surface area, pore volume and pore diameter were determined by BET to be 571 m²/g, 0.24 cc/g and 1.4 nm, respectively.

(N) Preparation of Microcomposite of TFESA and Silica

Tetramethyl orthosilicate (2 g), water (2.35 g) and 0.04 M HCl (0.025 g) were stirred together for 15 minutes to hydrolyze the tetraalkoxide. HCF₂CF₂SO₃H (2.37 g) was then added, and the mixture was stirred to gel (less than about 20 seconds). The resulting gel was left to dry in air in an uncovered beaker at room temperature for three days and then in a 70° C. oven under nitrogen for 24 hours. Drying of the composite was completed in a 100° C. vacuum oven for 24 hours. The composite comprised approximately 75% by weight of the acid relative to the weight of the silica.

(O) Preparation of Microcomposite of TFESA and Silica

Tetraethyl orthosilicate (14 g), water (12 g) and 1 M HCl (0.1 g) were stirred together for 2 hours to hydrolyze the tetraalkoxide. HCF₂CF₂SO₃H (1 g) was then added, and the mixture was stirred in an open beaker to gel (less than about two hours). The resulting gel was left to dry in air in an uncovered beaker at room temperature for 48 hours. Drying of the composite was completed in a 100° C. vacuum oven for 24 hours. The surface area, pore volume and pore diameter were determined by BET to be 342 m²/g, 0.16 cc/g and 1.9 nm, respectively.

(P) Preparation of Microcomposite of HFPSA and Silica

Tetramethyl orthosilicate (8 g), water (9.4 g) and 0.04 M HCl (0.1 g) were stirred together for 15 minutes to hydrolyze the tetraalkoxide. CF₃HCFCF₂SO₃H (1 g) was then added, and the mixture was stirred to gel (less than about two hours). The resulting gel was left to dry in air in an uncovered beaker at room temperature. Drying of the composite was completed in a 100° C. vacuum oven.

(Q) Preparation of 2-chloro-1.1.2-trifluoroethanesulfonic acid

Tetramethyl orthosilicate (8 g), water (9.4 g) and 0.04 M HCF (0.1 g) were stirred together for 15 minutes to hydrolyze the tetraalkoxide. HCFCICF₂SO₃H (1 g) was then added, and the mixture was stirred to gel (less than about two hours). The resulting gel was left to dry in air in an uncovered beaker at room temperature. Drying of the composite was completed in a 100° C. vacuum oven.

(R) Preparation of a Microcomposite of HCF₂CF₂SO₃H.H₂O on a Preformed Support

HCF₂CF₂SO₃H.H₂O (50 g) was added to 125 ml of diethyl ether. This mixture was added to 140 g of a spherical silica support (Silica Gel beads, 2-3 mm amorphous silicon dioxide beads, PQ Corporation, Valley Forge, Pa.) in a larger glass bottle. The bottle and contents were gently shaken for twenty minutes. The material was dried using a roto-vap at 35° C. under vacuum for 2 hours.

(S) Preparation of a Microcomposite of CF₃SO₃H (Triflic Acid) on a Preformed Support (Comparative Example)

CF3SO₃H (5.1 g) was added to 16.7 g of diethyl ether. This mixture was added to 16 g of a spherical silica support (Silica Gel beads, 2-3 mm amorphous silicon dioxide beads, PQ Corporation, Valley Forge, Pa.) in a larger glass bottle. The bottle and contents were gently shaken for twenty minutes. The material was dried using a roto-vap at 35° C. under vacuum for 2 hours.

Examples 1 to 7 illustrate the use of microcomposites of the invention in alkylation reactions.

### Example 1

### Comparison of the Catalytic Activity of HCF₂CF₂SO₃H.H₂O on Silica Versus CF₃SO₃H (Triflic Acid) on Silica

The catalytic activity of HCF₂CF₂SO₃H.H₂O on silica versus CF₃SO₃H (triflic acid) on silica were compared using an alkylation reaction.

(a) HCF₂CF₂SO₃H.H₂O on silica:

HCF₂CF₂SO₃H.H₂O on silica from Example 11 (1 g) was placed in an oven at 150° C., and dried overnight under vacuum. The dried material was rapidly added to a round bottomed flask containing 15 ml of p-xylene and 5 ml of dodecene under nitrogen. The flask and contents were heated at 100° C. with stirring. GC analysis at 2 hours showed that >95% of the dodecene had reacted to form the alkylated product.

(b) CF₃SO₃H (triflic acid) on silica:

CF₃SO₃H (triflic acid) on silica from Example 12 (1 g) was placed in an oven at 150° C., and dried overnight under vacuum. The dried material was rapidly added to a round bottomed flask containing 15 ml of p-xylene and 5 ml of dodecene under nitrogen. The flask and contents were heated at 100° C. with stirring. GC analysis at 2 hours showed that <1% of the dodecene had reacted to form the alkylated product.

### Example 2

### Alkylation of p-xylene with 1-dodecene in the Presence of the Microcomposite HCF₂CF₂SO₃H on Silica

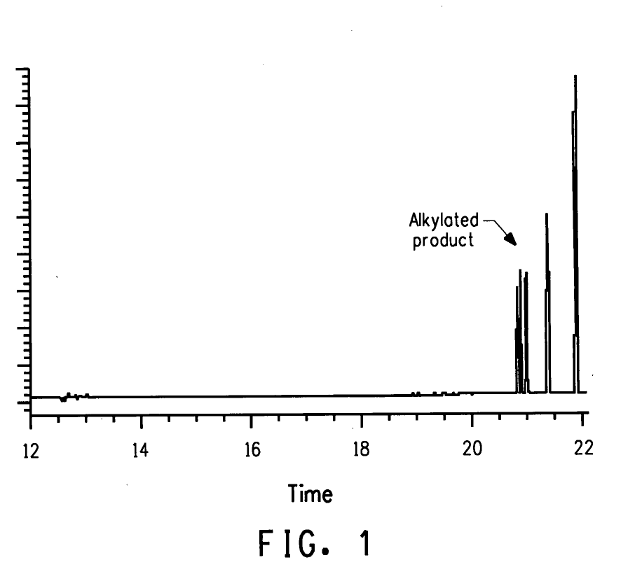

The acid catalyst HCF₂CF₂SO₃H supported on silica (24 wt % acid) was ground to a fine powder with a pestle and mortar. The finely ground powder (0.5 g) was weighed into a vial, dried at 150° C. under vacuum for at least four hours, and cooled under vacuum before transfer into a nitrogen atmosphere. The catalyst was loaded into a dried Schlenk flask, followed by the addition of anhydrous p-xylene (15 ml) and anhydrous 1-dodecene (5 ml). The flask was set up under a nitrogen blanket and stirred vigorously at 100° C. for 2 hours. GC analysis (see FIG. 1) of the products at 2 hours showed that >95% of the 1-dodecene was converted to the alkylated product.

### Example 3

### Alkylation of p-xylene with 1-dodecene in the Presence of HCF₂CF₂SO₃H (Comparative Example)

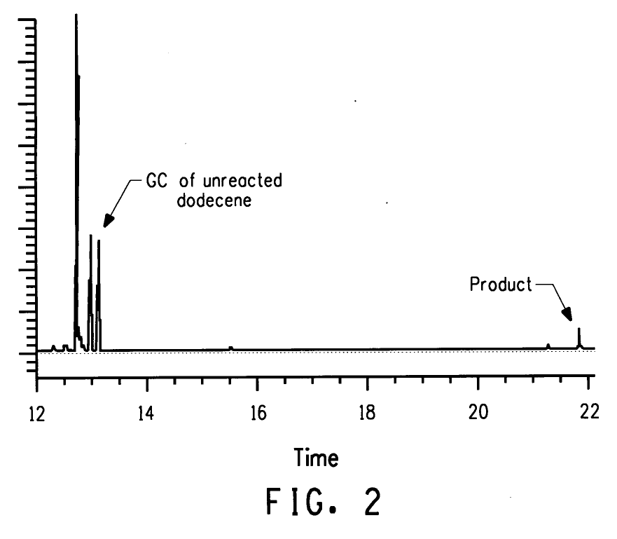

The acid catalyst HCF₂CF₂SO₃H (0.125) was loaded into a dried Schlenk flask under a nitrogen atmosphere, followed by the addition of anhydrous p-xylene (15 ml) and anhydrous 1-dodecene (5 ml). The flask was set up under a nitrogen blanket and stirred vigorously at 100° C. for 2 hours. GC analysis (see FIG. 2) of the products at 2 hours showed that <20% of the 1-dodecene was converted to the alkylated product.

### Example 4

### Alkylation of p-xylene with 1-dodecene with Recycle of the Microcomposite

The microcomposite HCF₂CF₂SO₃H supported on silica was ground to a fine powder with a pestle and mortar. The finely ground powder (0.5) was then weighed into a vial, dried at 150° C. under vacuum for at least four hours, and cooled under vacuum before transfer into a nitrogen atmosphere. The catalyst was loaded to a dried Schlenk flask, followed by the addition of anhydrous p-xylene (15 ml) and anhydrous 1-dodecene (5 ml). The flask was set up under a nitrogen blanket and stirred vigorously at 100° C. for 2 hours. Samples were withdrawn at 15 minutes, 1 hour and 2 hours, and diluted 1 to 20 in diethyl ether for GC analysis.

The mixture was cooled and transferred back to a nitrogen box. The solvent comprising unreacted p-xylene and 1-dodecene and the alkylated product was decanted and the solid was rinsed with fresh solvent mixture (15 ml p-xylene and 5 ml 1-dodecene). This was decanted and replaced with fresh solvent mixture. The flask was set up under a nitrogen blanket and stirred vigorously at 100° C. for 2 hours. GC analysis of the products at 2 hours showed that >96% of the 1-dodecene was converted to the alkylated product.

### Example 5

### Alkylation of p-xylene with 1-dodecene in the Presence of the Microcomposite HCF₂CF₂SO₃H on Silica

The acid catalyst HCF₂CF₂SO₃H supported on silica was ground to a fine powder with a pestle and mortar. The finely ground powder (0.5 g) was then weighed into a vial, dried at 150° C. under vacuum for at least four hours, and cooled under vacuum before transfer into a nitrogen atmosphere. The catalyst was loaded to a dried Schlenk flask, followed by the addition of anhydrous p-xylene (150 ml) and anhydrous 1-dodecene (50 ml). The flask was set up under a nitrogen blanket and stirred vigorously at 100° C. for 2 hours. Samples were withdrawn at 2 hours, 4 hours and 6.5 hours, and diluted 1 to 20 in diethyl ether for GC analysis. The reaction was stopped and left at room temperature for 3 days, restarted stirring at 100° C. for 7 hours, GC samples being drawn at 4.5 hours and 7 hours. GC analysis of the products at 2 hours showed that >90% of the 1-dodecene was converted to the alkylated product.

### Example 6

### Alkylation of p-xylene with 1-dodecene in the Presence the Microcomposite CF₃HCFCF₂SO₃H on Silica

The acid catalyst CF₃HCFCF₂SO₃H supported on silica was ground to a fine powder with a pestle and mortar. The finely ground powder (0.5 g) was then weighed into a vial, dried at 150° C. under vacuum for at least four hours, and cooled under vacuum before transfer into a nitrogen atmosphere. The catalyst was loaded into a dried Schlenk flask, followed by the addition of anhydrous p-xylene (15 ml) and anhydrous 1-dodecene (5 ml). The flask was set up under a nitrogen blanket and stirred vigorously at 100° C. for 2 hours. Samples were withdrawn at 15 minutes, 1 hour and 2 hours, and diluted 1 to 20 in diethyl ether for GC analysis. GC analysis of the products at 2 hours showed that >95% of the 1-dodecene was converted to the alkylated product.

### Example 7

### Alkylation of p-xylene with 1-dodecene in the Presence of the Microcomposite HCFCICF₂SO₃H on Silica

The acid catalyst HCFCICF₂SO₃H supported on silica was ground to a fine powder with a pestle and mortar. The finely ground powder (0.5 g) was then weighed into a vial, dried at 150° C. under vacuum for at least four hours, and cooled under vacuum before transfer into a nitrogen atmosphere. The catalyst was loaded into a dried Schlenk flask, followed by the addition of anhydrous p-xylene (15 ml) and anhydrous 1-dodecene (5 ml). The flask was set up under a nitrogen blanket and stirred vigorously at 100° C. for 2 hours. GC analysis of the products at 2 hours showed that >95% of the 1-dodecene was converted to the alkylated product.

1. A process for making at least one alkylated aromatic compound of the Formula: 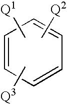
wherein:
a) Q₁ is H, —CH₃, —C₂H₅, or CH₃—CH—CH₃;
b) Q² is H, —CH₃ or —C₂H₅; and
c) Q³ is —C₂H₅ or C₃ to C₁₈ straight chain alkyl group having therein a single CH group, the carbon atom of which is bonded to the aromatic compound;
by a process comprising reacting a C₂ to C₁₈ straight-chain monoolefin with an aromatic compound of the Formula: 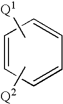
wherein Q₁ and Q² are as defined above;
in the presence of at least one porous microcomposite comprising at least one fluorinated sulfonic acid and silica made by a process comprising the steps of:
(a) contacting, in the presence of water:
(1) at least one silica precursor;
(2) at least one fluorosulfonic acid selected from the group consisting of:
(i) 1,1,2,2-tetrafluoroethanesulfonic acid;
(ii) 1,1,2-trifluoro-2-(perfluoroethoxy)ethanesulfonic acid;
(iii) 1,1,2-trifluoro-2-(trifluoromethoxy)ethanesulfonic acid;
(iv) 1,1,2-trifluoro-2-(perfluoropropoxy)ethanesulfonic acid;
(v) 1,1,2,3,3,3-hexafluoropropanesulfonic acid; and
(vi) 2-chloro-1,1,2-trifluoroethanesulfonic acid; and optionally at least one inorganic acid; and
(3) optionally, a non-reacting solvent; to form a mixture;
(b) aging the mixture to form a gelled mixture; and
(c) drying the gelled mixture to remove substantially all water and non-reacting solvent, if any, therein;
said reacting being carried out at a temperature between about 25° C. and about 200° C., and a pressure between atmospheric pressure and that pressure required to maintain the reactants in a liquid state.

2. The process of claim 1 wherein Q₁ and Q² are H.

3. The process of claim 1 wherein the aromatic compound is benzene, xylene, ethyl benzene or isopropyl benzene.

4. The process of claim 1 wherein the silica precursor is selected from the group consisting of:
(i) silicon alkoxides of the Formula Si(OR)₄, wherein R is —CH₃, —C₂H₅, or C3 to C6 straight-chain or branched alkyl;
(ii) silicon tetrachloride;
(iii) CH₃Si(OCH₃)₃;
(iv) PhSi(OCH₃)₃, where Ph is phenyl;
(v) (CH₃)₂Si(OCH₃)₂; and (vi) polysilicic acid.

5. The process of claim 1 wherein the inorganic acid is selected from the group consisting of hydrochloric acid, sulfuric acid and nitric acid.

6. The process of claim 1 wherein the porous microcomposite is used at a concentration of from about 0.01% to about 20% by weight of the reaction solution comprising the aromatic compound and the monoolefin.

7. The process of claim 1 the temperature of said reacting is about 25° C. and the pressure is atmospheric pressure.

8. The process of claim 1 wherein the molar ratio of the aromatic compound to the monoolefin at the start of the reaction is at least about 3:1.

9. A process for making at least one alkylated aromatic compound of the Formula: 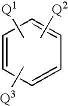
wherein:
a) Q₁ is H, —CH₃, —C₂H₅, or CH₃—CH—CH₃;
b) Q² is H, —CH₃ or —C₂H₅; and
c) Q³ is —C₂H₅ or C₃ to C₁₈ straight chain alkyl group having therein a single CH group, the carbon atom of which is bonded to the aromatic compound;
by a process comprising reacting a C₂ to C₁₈ straight-chain monoolefin with an aromatic compound of the Formula: 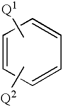
wherein Q₁ and Q² are as defined above;
in the presence of at least one porous microcomposite comprising at least one fluorinated sulfonic acid and silica made by a process comprising the steps of:
(a) contacting at least one fluorosulfonic acid selected from the group consisting of:
(i) 1,1,2,2-tetrafluoroethanesulfonic acid;
(ii) 1,1,2-trifluoro-2-(perfluoroethoxy)ethanesulfonic acid;
(iii) 1,1,2-trifluoro-2-(trifluoromethoxy)ethanesulfonic acid;
(iv) 1,1,2-trifluoro-2-(perfluoropropoxy)ethanesulfonic acid;
(v) 1,1,2,3,3,3-hexafluoropropanesulfonic acid; and
(vi) 2-chloro-1,1,2-trifluoroethanesulfonic acid;
optionally in a non-reacting solvent, with a preformed porous silica support;
(b) drying the acid-impregnated porous silica to remove therefrom substantially all of the non-reacting solvent and water, if any, contained therein;
said reacting being carried out at a temperature between about 25° C. and about 200° C., and a pressure between atmospheric pressure and that pressure required to maintain the reactants in a liquid state.

10. The process of claim 9 wherein Q₁ and Q² are H.

11. The process of claim 9 wherein the aromatic compound is benzene, xylene, ethyl benzene or isopropyl benzene.

12. The process of claim 9 wherein the silica precursor is selected from the group consisting of:
(i) silicon alkoxides of the Formula Si(OR)₄, wherein R is —CH₃, —C₂H₅, or C3 to C6 straight-chain or branched alkyl;
(ii) silicon tetrachloride;
(iii) CH₃Si(OCH₃)₃;
(iv) PhSi(OCH₃)₃, where Ph is phenyl;
(v) (CH₃)₂Si(OCH₃)₂; and
(vi) polysilicic acid.

13. The process of claim 9 wherein the aromatic compound is benzene, xylene, ethyl benzene or isopropyl benzene.

14. The process of claim 9 wherein the porous microcomposite is used at a concentration of from about 0.01% to about 20% by weight of the reaction solution comprising the aromatic compound and the monoolefin.

15. The process of claim 9 wherein the temperature of said reacting is about 25° C. and the pressure is atmospheric pressure.

16. The process of claim 9 wherein the molar ratio of the aromatic compound to the monoolefin at the start of the reaction is at least about 3:1.
